(19) United States
(12) Patent Application Publication
(10) Pub. No.: US 2007/0021648 A1
(43) Pub. Date: Jan. 25, 2007

(54) TRANSLUMINAL SHEATH HUB

(76) Inventors: Jay Lenker, Laguna Beach, CA (US); Edward J. Nance, Corona, CA (US); Joseph Bishop, Menifee, CA (US); George F. Kick, Casa Grande, AZ (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/427,729

(22) Filed: Jun. 29, 2006

Related U.S. Application Data
(60) Provisional application No. 60/695,790, filed on Jun. 29, 2005.

(51) Int. Cl. A61F 2/00

(52) U.S. Cl. 600/29

(57) ABSTRACT

Disclosed is a hub for a transluminal sheath. The hub provides a handle for grasping the sheath, provides connections for fluid inlet and outlet lines, and provides for attaching mechanisms between the sheath and a dilator. The hub can be used on a non-radially expandable sheath, or it can be used on a sheath having a radially expandable configuration. In an exemplary application, the hub is fitted to a sheath, which provides access for a diagnostic or therapeutic procedure such as ureteroscopy or stone removal.

### PRIORITY INFORMATION

This application claims the priority benefit under 35 U.S.C. § 119(e) of Provisional Application 60/695,790, filed Jun. 29, 2005, the entirety of which is hereby incorporated by reference herein.

### BACKGROUND OF THE INVENTION

1. Field of the Invention

The invention relates to medical devices and, more particularly, to medical devices for transluminally accessing body lumens and cavities.

2. Description of the Related Art

A wide variety of diagnostic or therapeutic procedures involves the introduction of a device through a natural access pathway such as a body lumen or cavity. A general objective of such access systems, which have been developed for this purpose, is to minimize the cross-sectional area of the access lumen, while maximizing the available space for the diagnostic or therapeutic instrumentation. These procedures are especially suited for the urinary tract of the human or other mammal. The urinary tract is relatively short and substantially free from the tortuosity found in many endovascular applications.

Ureteroscopy is an example of one type of therapeutic interventional procedure that relies on a natural access pathway, which is the urethra, the bladder, which is a body cavity, and the ureter, another body lumen. Ureteroscopy is a minimally invasive procedure that can be used to provide access to the upper urinary tract, specifically the ureter and kidney. Ureteroscopy is utilized for procedures such as stone extraction, stricture treatment, or stent placement. Other types of therapeutic interventional procedures suitable for use with expandable sheath technology include endovascular procedures such as introduction of cardiac valve replacements or repair devices via a percutaneous access to the vasculature. Gastrointestinal procedures, again percutaneously performed, include dilation of the common bile duct and removal of gallstones.

To perform a procedure in the ureter, a cystoscope is placed into the bladder through the urethra, a body lumen. A guidewire is next placed, through the working channel of the cystoscope and under direct visual guidance, into the target ureter. Once guidewire control is established, the cystoscope is removed and the guidewire is left in place. A ureteral sheath or catheter is next advanced through the urethra over the guidewire, through the bladder and on into the ureter. The guidewire may now be removed to permit instrumentation of the ureteral sheath or catheter. A different version of the procedure involves leaving the guidewire in place and passing instrumentation alongside or over the guidewire. In yet another version of the procedure, a second guidewire or “safety wire” may be inserted into the body lumen or cavity and left in place during some or all of the procedure.

Current techniques involve advancing a flexible, 10 to 18 French, ureteral sheath or catheter with integral flexible, tapered obturator over the guidewire. Because axial pressure is required to advance and place each catheter, care must be taken to avoid kinking the sheath, catheter, or guidewire during advancement so as not to compromise the working lumen of the catheter through which instrumentation, such as ureteroscopes and stone extractors, can now be placed. The operator must also exercise care to avoid advancing the sheath or catheter against strictures or body lumen or cavity walls with such force that injury occurs to said body lumen or cavity walls.

One of the issues that arise during ureteroscopy is the need to grasp the proximal end of the sheath. An optimized hub facilitates such operator interface. A hub that is too large in diameter, too small in diameter, or too difficult to grip is suboptimal. Another issue that arises during ureteroscopy is the attachment between the sheath and a dilator or obturator inserted therethrough. The sheath and obturator should not inadvertently come apart or separate during sheath introduction but should be able to be selectively separated at the discretion of the operator, following introduction and placement. Furthermore, the hub needs to be able to guide instrumentation inserted into the sheath so that such introduction of instrumentation is not difficult or tedious. Additionally, the hub needs to provide for secure and reversible connection of flushing lines, which guide fluid into, or out of, the sheath. Sheath hubs available today do not have secure connections to the dilator hub and are often too large for easy grasping.

Additional information regarding ureteroscopy can be found in Su, L, and Sosa, R. E., Ureteroscopy and Retrograde Ureteral Access, Campbell's Urology, 8th ed, vol. 4, pp. 3306-3319 (2002), Chapter 97. Philadelphia, Saunders, and Moran, M. E., editor, Advances in Ureteroscopy, Urologic Clinics of North America, vol. 31, No. 1 (February 2004).

A need therefore remains for improved access technology, which offers improved grip by the user and for secure attachment to obturators, dilators, and fluid lines. Ideally, the hub technology allows a sheath to be transluminally and grasped by an operator using their thumb and index finger. Ideally, the sheath would be able to enter a vessel or body lumen and be able to pass instruments through a central lumen that was 10 to 18 French. The sheath could be non-expandable, or it could be expandable to permit a smaller introduction size than the final operational size. The sheath and hub would also be maximally visible under fluoroscopy and would be relatively inexpensive to manufacture. The sheath or catheter would be kink resistant and minimize abrasion and damage to instrumentation being passed therethrough.

### SUMMARY OF THE INVENTION

Accordingly, one embodiment of the present invention comprises a transluminal access sheath for insertion into a urethra by a person having a pair of adjacent fingers. The access sheath can comprise an elongate tube having a lumen extending between a proximal end and a distal end, the elongate tube having a distal portion and a proximal portion. A removable inner member can be disposed within the lumen of the elongate tube. A hub can be coupled to the proximal end of the elongate tube. The hub can comprises a distally facing surface and a proximally facing surface. The distally facing surface can form at least in part a straight cone, sized and configured to receive adjacent fingers of the user. The proximally facing surface can form a straight taper configured to funnel instrumentation into the lumen.

For purposes of summarizing the invention, certain aspects, advantages and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein. These and other objects and advantages of the present invention will be more apparent from the following description taken in conjunction with the accompanying drawings.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings, reference numbers are re-used to indicate correspondence between referenced elements.

FIG. 1 is a longitudinal cross-sectional view of the proximal end of a transluminal sheath comprising a hub according to an embodiment of the present invention.

FIG. 2 is a longitudinal cross-sectional view of the proximal end of a transluminal sheath comprising a hub according to another embodiment of the present invention.

FIG. 3 is a longitudinal cross-sectional view of the proximal end of a transluminal sheath comprising a hub according to another embodiment of the present invention;

FIG. 4 is a longitudinal cross-sectional view of the proximal end of a transluminal sheath comprising a hub according to another embodiment of the present invention;

FIG. 5 is a longitudinal cross-sectional view of the proximal end of a transluminal sheath comprising a hub according to another embodiment of the present invention

FIG. 6 is a longitudinal cross-sectional view of the proximal end of a transluminal sheath comprising a hub according to another embodiment of the present invention.

FIG. 7A is a cross-sectional illustration of an embodiment of a radially expandable transluminal catheter or sheath comprising a tube that is folded, at its distal end in longitudinal creases, a balloon dilator, and an outer retaining sleeve, the sheath tube and dilator being in their radially collapsed configuration.

FIG. 7B is a partial cross-sectional illustration of the radially expandable transluminal sheath of FIG. 7A, wherein the sheath and the dilator are in their radially expanded configuration.

FIG. 7C illustrates a side view of the radially expanded transluminal sheath of FIG. 7B, wherein the dilator has been removed, according to an embodiment of the invention.

FIG. 8A illustrates a side cutaway view of another embodiment of a radially collapsed sheath comprising an expandable distal region with one or more longitudinal folds and a malleable coil reinforcing layer embedded within the distal region.

FIG. 8B illustrates the sheath of FIG. 6A, with cutaway sections, wherein the balloon has expanded the distal region of the sheath to its fully expanded configuration.

FIG. 9A illustrates a lateral cross-section of an embodiment of a sheath tube configured with discreet thin areas, running longitudinally along the tube.

FIG. 9B illustrates a lateral cross-section of the sheath tube of FIG. 10A which has been folded at the thin areas to create a smaller diameter tube.

FIG. 9C illustrates a lateral cross-section of the sheath tube of FIG. 10B, which has been folded down over a balloon, which has further been folded into four flaps and has been compressed against its central tubing.

FIG. 9D illustrates a lateral cross-section of an embodiment of a sheath tube comprising an inner lubricious layer, a reinforcing layer, an intermediate elastomeric layer, and an outer lubricious layer.

FIG. 9E illustrates a lateral cross-section of an embodiment of an expandable sheath tube comprising a double longitudinal fold.

### DETAILED DESCRIPTION OF THE PREFERRED EMBODIMENTS

In the embodiments described below reference will be made which is to a “catheter” or a “sheath”, which can comprise a generally axially elongate hollow tubular structure having a proximal end and a distal end. The axially elongate structure can include a longitudinal axis and an internal through lumen that extends from the proximal end to the distal end for the passage of instruments, implants, fluids, tissue, or other materials. While in many of the embodiments described herein the tubular structure has a generally round or circular cross-section, in modified embodiments, the tubular structure can have a non-round (e.g., square) or non-circular (e.g., oval) cross-section. The axially elongate hollow tubular structure can be generally flexible and capable of bending, to a greater or lesser degree, through one or more arcs in one or more directions perpendicular to the main longitudinal axis. As is commonly used in the art of medical devices, the proximal end of the device is that end that is closest to the user, typically a surgeon or interventionalist. The distal end of the device is that end closest to the patient or that is first inserted into the patient. A direction being described as being proximal to a certain landmark will be closer to the surgeon, along the longitudinal axis, and further from the patient than the specified landmark. The diameter of a catheter is often measured in “French Size” which can be defined as 3 times the diameter in millimeters (mm). For example, a 15 French catheter is 5 mm in diameter. The French size is designed to approximate the circumference of the catheter in mm and is often useful for catheters that have non-circular cross-sectional configurations.

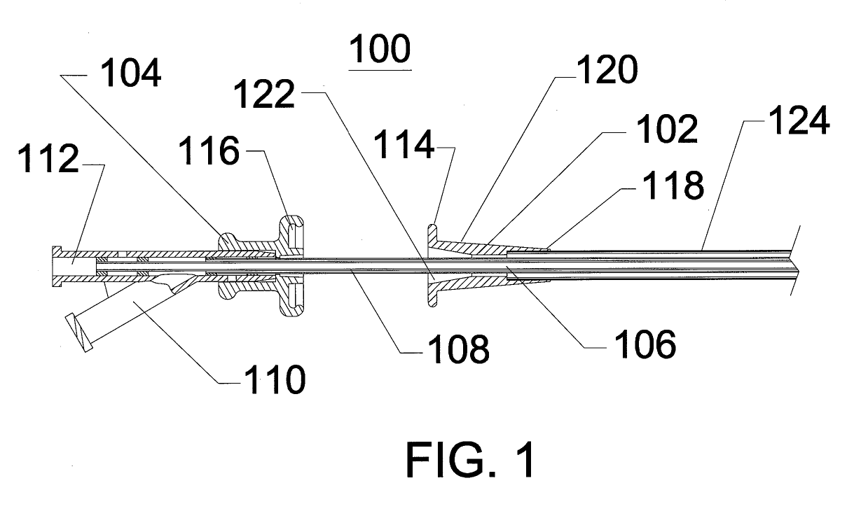

FIG. 1 is a longitudinal cross-sectional view of the proximal end of one embodiment of a transluminal sheath system 100. In this embodiment, the system 100 comprises a hub 102 having a gentle proximally facing straight conical taper 120 and a gentle distally facing straight conical internal taper 122 as well as a dilator hub 104 that mates to the exterior perimeter 114 of the sheath hub 102. The sheath hub 102 further comprises a distal end that is small in diameter and can slip into a lumen, a mating feature 114 a straight conical distally facing taper 120, and a sheath tube 106. The proximal end of the sheath tube 106 is disposed so that it can slip into the body lumen when the distal end 118 slips into the body lumen. The distal end 118 of the sheath hub 102 can be smooth, tapered, and comprises a fillet or chamfer to present an atraumatic edge to the body lumen. The distal end 118 does not comprise a flange or large diameter edge that would prevent the distal end of the hub from entering the body lumen. The dilator hub 104 comprises a mating detent 116 that releasably is affixed to the mating feature 114 on the sheath hub 102. The mating features can be grooves and corresponding bumps, latches, quick connects, bayonet mounts, threads, and the like. The dilator hub 104 further comprises a guidewire port 112 and an inflation port 110, which can be both luer ports in one embodiment or luer lock ports in another embodiment. The sheath hub 102 and the dilator hub 104 are preferably machined, CNC machined, molded, or insert molded over the tubing 124 and 108 respectively. The sheath hub 102 and dilator hub 104 are fabricated from materials such as, but not limited to, polyethylene, polypropylene, polyurethane, polyvinyl chloride, acrilonitrile butadiende styrene, polycarbonate, polyamide, polyimide, stainless steel, and the like. The two hubs 102 and 104 can advantageously be fabricated from dissimilar materials to prevent blocking.

The system 100 and tubing 124 can be coupled to, integrally formed and/or used with a variety of non-expandable or expandable components, which form a distal portion (not shown) of the systems described herein. For example, those of skill in the art will recognize that the system and hubs 102, 104 described herein can be used in combination with the sheaths/systems described in U.S. patent application Ser. No. 10/884,017, filed Jul. 2, 2004 (Publication No. 2005-0125021), U.S. patent application Ser. No. 10/841,799, filed May 7, 2004 (Publication No. 2005-0222576), U.S. patent application Ser. No. 10/841,965, filed May 7, 2004 (Publication No. 2005-0209627), U.S. patent application Ser. No. 11/223,897, filed Sep. 9, 2005 (Publication No. 2006-0135981), U.S. patent application Ser. No. 11/313,400, filed Dec. 21, 2005 (Publication No.), and U.S. patent application Ser. No. 11/199,566, filed Sep. 9, 2005 (Publication No. 2006-0052750), the entire contents of which are hereby incorporated by reference herein.

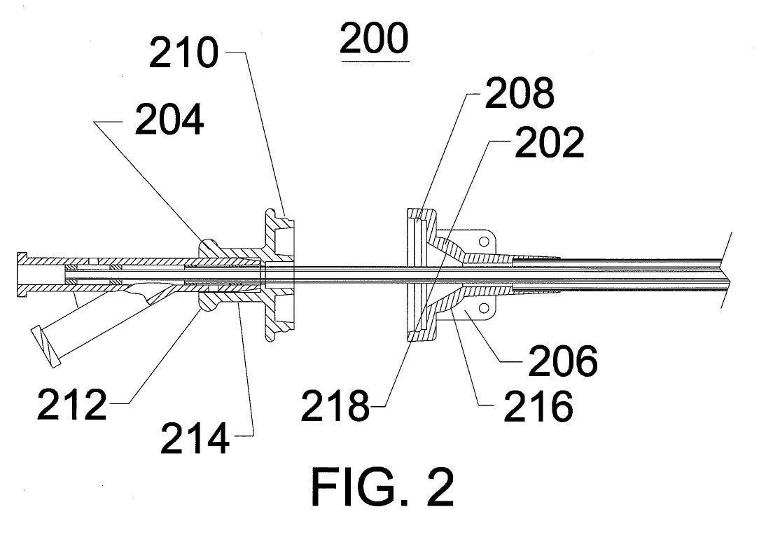

FIG. 2 is a longitudinal cross-sectional view of another embodiment of a proximal end of a transluminal sheath system 200 comprising a sheath hub 202 having a proximally facing convex conical taper 216 and a gentle distally facing straight conical internal taper 218. The sheath system 200 comprises a dilator hub 204 that further comprises external circumferential ridges 210 that mate to circumferential grooves 208 disposed on the interior perimeter of the proximal end of the sheath hub 202. The sheath hub 202 further comprises grommets 206 that can be affixed or integrally formed with the sheath hub 202 to permit sutures or clips to be attached thereto. The dilator hub 204 can further comprise a gripping surface 214 and a gripping ridge 212 to facilitate gripping the dilator hub 204 with the thumb and index finger. The external ridges 210 on the dilator hub 204 and the internal grooves 208 on the sheath hub can furthermore be reversed but the illustrated embodiment is preferred to minimize the need for secondary operations following the molding process.

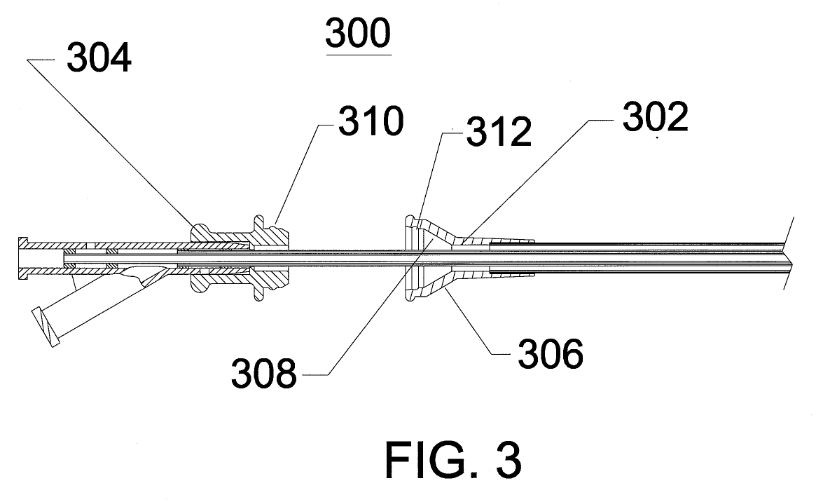

FIG. 3 is a longitudinal cross-sectional view of the proximal end of another embodiment transluminal sheath system 300 comprising a small diameter sheath hub 302 having a proximally facing two-stage straight conical taper 306 and a gentle distally facing straight conical internal taper 308. The sheath system 300 can comprise a dilator hub 304 with circumferential external ridges 310 that mate to circumferential interior grooves 312 on the sheath hub 302. The sheath system 300 of this embodiment can have an overall diameter at the hub 302 and 304 not exceeding 0.7 inches.

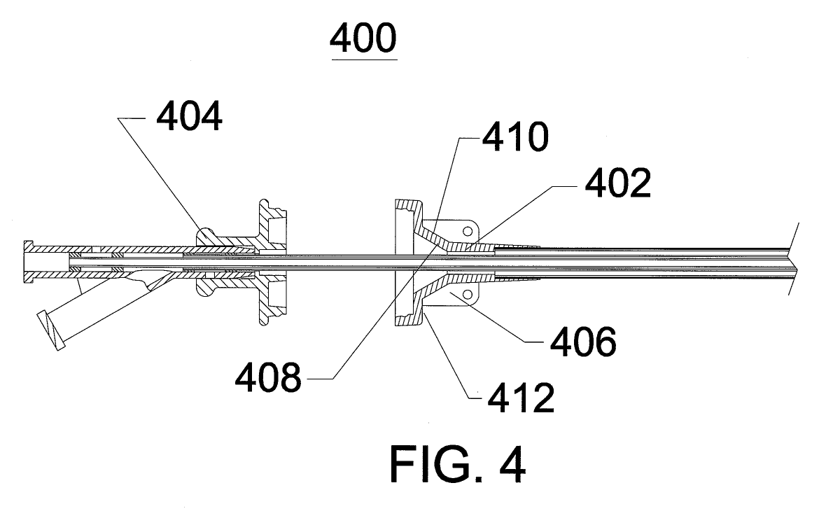

FIG. 4 is a longitudinal cross-sectional view of the proximal end of another embodiment transluminal sheath system 400 comprising a large diameter sheath hub 402 having a proximally facing two-stage straight conical taper 410, a lateral wall 412, one or more optional grommets 406 disposed on integrally molded fins, and a gentle distally facing straight conical internal taper 408. The sheath system 400 can comprise a dilator hub 404 that mates to the interior perimeter of the hub of the sheath in the same or similar way as the sheath system 200 of FIG. 2. However, in this embodiment, there is no distally facing convex curve 202 in longitudinal cross-section. Otherwise, this sheath system 400 is the similar as the sheath system 200.

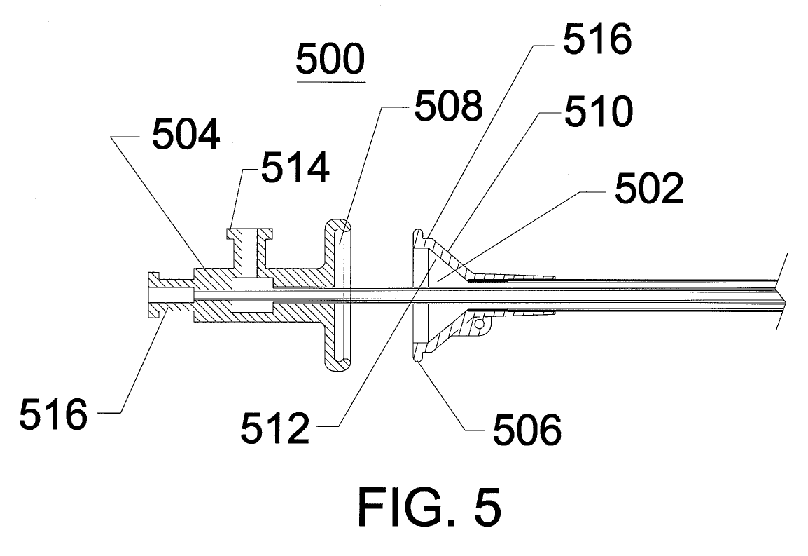

FIG. 5 is a longitudinal cross-sectional view of the proximal end of a another embodiment of a transluminal sheath system 500 comprising a large diameter sheath hub 502 having a proximally facing two-stage straight conical taper 510 with a proximal flat region 516, external circumferentially oriented mating ridges 506, and a distally facing straight conical internal taper 512. The sheath system 500 can comprise a dilator hub 504 that comprises a guidewire port 516, an inflation port 514, and internal circumferentially oriented grooves 508 that mate to the exterior ridges 506 of the sheath hub 502. The guidewire port 516 and the inflation port 514 are integrally molded into the dilator hub 504 and are not separately bonded, welded, or otherwise affixed thereto. Either this type of integral construction, lending itself to insert molding, or the composite construction shown in FIG. 1, for example, are suitable for use in a transluminal sheath.

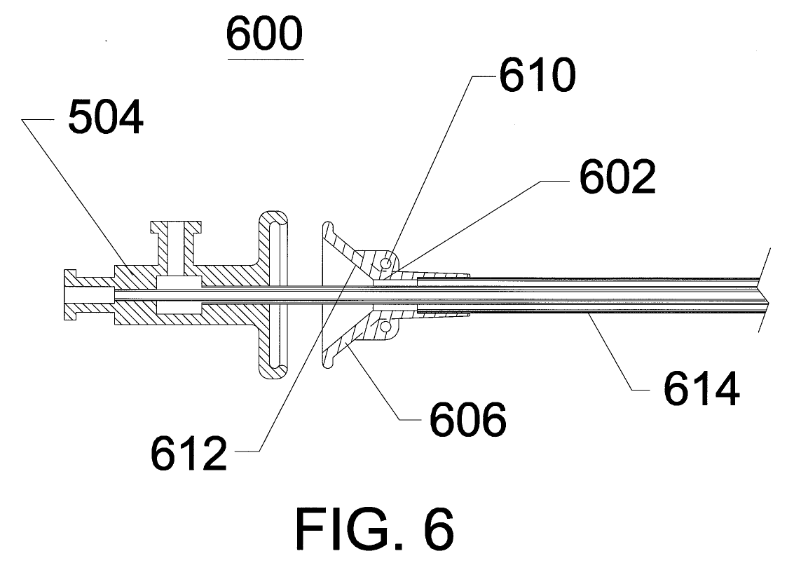

FIG. 6 is a longitudinal cross-sectional view of the proximal end of another embodiment transluminal sheath system 600 comprising a large diameter sheath hub 602 having a proximally facing two-stage straight conical taper 606 without the proximal flat region 516 shown in FIG. 5, and a distally facing straight conical internal taper 612 as well as an integrally molded dilator hub 504 that mates to the exterior perimeter of the sheath hub 602. Other than the absence of the flat region 516, the sheath hub 602 is substantially the same as the sheath hub 502 of FIG. 5. The dilator hub 504 can be the same as or similar as that shown in FIG. 5.

In an embodiment, the dilator hub 604 is keyed so that when it is interfaced to, or attached to, the sheath hub 602, the two hubs 604 and 602 cannot rotate relative to each other. The anti-rotation keys or features could include mechanisms such as, but not limited to, one or more keyed tab on the dilator hub 804 and one or more corresponding keyed slot in the sheath hub 602. Axial separation motion between the dilator hub 604 and the sheath hub 602 easily disengages the two hubs 604 and 602 while rotational relative motion is prevented by the sidewalls of the tabs and slots. A draft angle on the sidewalls of the tabs and the slots further promotes engagement and disengagement of the anti-rotation feature. In another embodiment, the sheath hub 602 is releaseably affixed to the dilator hub 604 so the two hubs 604 and 602 are coaxially aligned and prevented from becoming inadvertantly disengaged or separated laterally. In this embodiment, the two hubs 604 and 602 are connected at a minimum of 3 points, which prevent lateral relative motion in both of two substantially orthogonal axes. In a preferred embodiment, the two hubs 604 and 602 are engaged substantially around their full 360-degree perimeter. Manual pressure is sufficient to snap or connect the two hubs 604 and 602 together as well as to separate the two hubs 604 and 602.

In an embodiment, the distal end of the sheath hub 602 is configured to taper into the sheath tubing 614 so that the sheath hub 602 distal end and the proximal end of the sheath tubing 614 can be advanced, at least partly, into the urethra or urethral meatus or other body lumen without causing tissue damage. The sheath hub 602 serves as the handle for the sheath system 600 and is generally a cylinder of revolution with certain changes in outside diameter moving from distal to proximal end. In an embodiment, the distal facing surface 606 of the sheath hub 602 can define a cone tapering inward moving increasingly distally. The cone, in longitudinal cross-section, can be characterized by two exterior walls, symmetrically disposed about a centerline, each of said exterior walls being curvilinear and describing a concave outline. In a preferred embodiment, the exterior outline of the distal surface 606 of the sheath hub 602 can describe a linear outline, with surfaces running generally parallel to the longitudinal axis of the sheath tubing 614 and other surfaces running generally perpendicular to the longitudinal axis of the sheath tubing 614. In this preferred embodiment, there are no curvilinear axial cross-sectional outlines except at regions of fillets or other rounding to substantially eliminate any sharp edges that could cut through gloves or fingers. The proximally facing surface 612 of the sheath hub 602 can be curvilinear and flared with a longitudinal cross-section outline appearing like the internal surface of a bell, such shape acting as a funnel for instrumentation. In this embodiment, the axial cross-sectional view of the distally facing surface 606 describes two interior walls, symmetrically disposed about a centerline, each of the walls being convex when viewed from the proximal end of the sheath 600. In a preferred embodiment, the proximally facing surface 612 of the sheath hub 602 can appear substantially linear with edges that are oriented substantially perpendicular to the longitudinal axis of the sheath tubing 614. The access through the proximal surface 612 of the sheath hub 602 to the inner lumen of the sheath 600, can be curvilinear and flared, or it can be linear and describe a lumen that is generally parallel to the longitudinal axis. In another embodiment, the access port through the proximal end 612 of the sheath hub 602 can comprise a straight taper, such as a 6 percent Luer taper to allow for sealing with other devices inserted therein or to allow for ease of device insertion. The amount of end taper can vary between 1½ degrees and 20 degrees between each side and the longitudinal axis of the sheath 600. The maximum outer diameter of the sheath hub 602 can be between 0.25 and 2.0 inches, with a preferred range of between 0.5 and 1.0 inches. The sheath hub 602 can be sized so that at least half a finger diameter is cradled by each side of the flange of the hub 602. The distally facing surface 606 of the sheath hub 602 can furthermore be shaped to have substantially the same curve radius as a finger, so as to be received, or grasped, between two fingers of the hand, cigarette style, like the technique used for control of cystoscopes. In another embodiment, the sheath hub 602 can be sized and configured to be grasped between a thumb and finger, like a pencil or catheter, where there are no features or curves on the distally facing surface 606 of the sheath hub 602 to approximately match or conform to the shape or diameter of two fingers.

The proximal sheath tube 614 can be affixed to the sheath hub 602 by insert molding, bonding with adhesives, welding, or the like. The sheath system 600 can further comprise a valve operably connected to the sheath hub 602 and a hemostatic valve operably connected to the dilator hub 604. The valve is a duckbill valve, one-way valve, or other sealing-type valve capable of opening to a large bore and yet closing around instrumentation such as the dilator shaft. The valve seals against fluid loss from the internal lumen of the sheath 600 while the dilator hub 604 is connected to the sheath hub 602 after the dilator shaft has been removed from the sheath 600. The valve can be integral to the sheath hub 602, it can be welded or adhered to the sheath hub 602, or it can be affixed by a Luer fitting or other quick connect fitting. The hemostatic valve can be a Tuohy-Borst valve or other valve capable of sealing against a guidewire or small instrument and remain sealed after removal of said guidewire or small instrument. The hemostatic valve may further comprise a tightening mechanism (not shown) to enhance sealing against guidewires or against an open lumen. The hemostatic valve can be integral to the dilator hub 604, it can be welded or adhered to the dilator hub 604, or it can be affixed by a Luer fitting or other quick connect fitting. The valves are generally fabricated from polymeric materials and have soft resilient seal elements disposed therein. The hemostatic valve is intended to minimize or prevent blood loss from vessels at systemic arterial pressure for extended periods of time. The valve is intended to minimize or eliminate blood loss when instrumentation of various diameters is inserted therethrough.

In another embodiment, the distal end of the sheath hub can be tapered to an increasingly small diameter moving distally so that the distal end, as well as the proximal end of the sheath tube, can slip substantially within a body vessel or lumen, for example a urethra. The proximal port of the sheath hub can be straight, it can be tapered, or it can have a straight taper to facilitate sealing with the dilator distal taper. The taper angle can be between 1 degree and 20 degrees on each side. The dilator hub knob is integral to the dilator hub and provides an enlargement that can be gripped by the user to facilitate separation of the dilator hub from the sheath hub. The dilator hub knob also can be used between the thumb and a finger or between two fingers to advance the entire assembly or remove the assembly from the patient.

Accordingly, a transluminal access sheath with integral hub can be provided. In one embodiment, the access sheath is used to provide access to the ureter, kidney, or bladder. In such an embodiment, the sheath can have an introduction outside diameter that ranged from 4 to 24 French with a preferred range of 6 to 18 French. The ability to pass the large instruments through a catheter introduced with a small outside diameter can be derived from the ability to expand the distal end of the catheter to create a larger through lumen. The expandable distal end of the catheter can comprise 75% or more of the overall working length of the catheter. The proximal end of the sheath is generally larger to provide for pushability, control, and the ability to pass large diameter instruments therethrough.

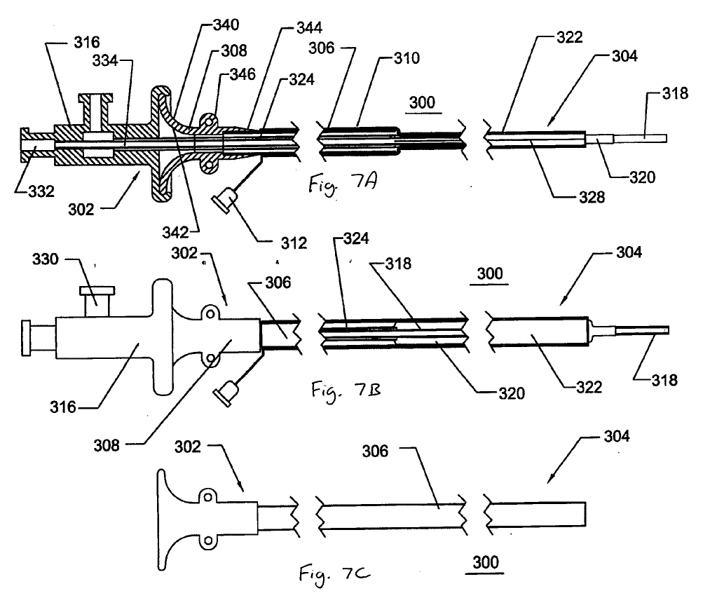

With reference now to FIG. 7A illustrates a longitudinal view of an embodiment of an expandable transluminal sheath 300, which is used to illustrate an embodiment of a distal end of the sheath that can be expandable. In this figure, the front (distal) section of the sheath 300 is depicted in exterior view and not in cross-section. The proximal region 302 and the central region are shown in longitudinal cross-section. The transluminal sheath 300 comprises a proximal end 302 and a distal end 304. In the illustrated embodiment, the proximal end 302 further comprises a proximal sheath tube 306, a sheath hub 308, an optional sleeve 310, an optional sleeve grip 312, an inner catheter shaft 318, an outer catheter shaft 324, and a catheter hub 316. The catheter hub 316 further comprises the guidewire access port 332. The catheter shaft 318 further comprises a guidewire lumen 334. The distal end 304 further comprises a distal sheath tube 322, the inner catheter shaft 318, and a balloon 320. The distal sheath tube 322 is folded longitudinally into one, or more, creases 328 to reduce the tubes 322 cross-sectional profile. The sheath hub 308 further comprises a distally facing surface 340, a proximally facing surface 342, a tapered distal edge 344, and a tie-down grommet 346.

Referring to FIG. 3A, the proximal end 302 generally comprises the proximal sheath tube 306 that can be permanently affixed or otherwise coupled to the sheath hub 308. The optional sleeve 310 is tightly wrapped around the proximal sheath tube 306 and is generally able to be split lengthwise and be removed or disabled as a restraint by pulling on the optional sleeve grip 312 that is affixed to the sleeve 310. The optional sleeve 310 is preferably fabricated from transparent material, or material with a color other than that of the sheath 300, and is shown so in FIGS. 3A and 3B. The proximal end further comprises the inner catheter shaft 318, the outer catheter shaft 324, and the catheter hub 316. The catheter hub 316 is integrally molded with, welded to, bonded or otherwise coupled, to the guidewire port 332. The dilator, or catheter, hub 316 allows for gripping the dilator and it allows for expansion of the dilatation balloon 320 by pressurizing an annulus between the inner catheter shaft 318 and the outer catheter shaft 324, said annulus having openings into the interior of the balloon 320. The balloon 320 is preferably bonded, at its distal end, either adhesively or by fusion, using heat or ultrasonics, to the inner catheter shaft 318. The proximal end of the balloon 320 is preferably bonded or welded to the outer catheter shaft 324. In another embodiment, pressurization of the balloon 320 can be accomplished by injecting fluid, under pressure, into a separate lumen in the inner or outer catheter shafts 318 or 324, respectively, said lumen being operably connected to the interior of the balloon 320 by openings or scythes in the catheter tubing. Such construction can be created by extruding a multi-lumen tube, rather than by nesting multiple concentric tubes. The distal end 304 generally comprises the distal sheath tube 322 which is folded into creases 328 running along the longitudinal axis and which permit the area so folded to be smaller in diameter than the sheath tube 306. The inner catheter shaft 318 comprises a guidewire lumen 334 that may be accessed from the proximal end of the catheter hub 316 and preferably passes completely through to the distal tip of the catheter shaft 318. The guidewire lumen 334 is able to slidably receive guidewires up to and including 0.038-inch diameter devices.

As mentioned above, the proximal end of the sheath 300 comprises the sheath hub 308 and the dilator hub 316. In one embodiment, the dilator hub 316 is keyed so that when it is interfaced to, or attached to, the sheath hub 308, the two hubs 308 and 316 cannot rotate relative to each other. This is beneficial so that the balloon 320 or the dilator shaft 318 do not become twisted due to inadvertent rotation of the dilator hub 316 relative to the sheath hub 308. A twisted balloon 320 has the potential of not dilating fully because the twist holds the balloon 320 tightly to the dilator shaft 318 and prevents fluid from fully filling the interior of the balloon 320. Twisting of the dilator shaft 318 or balloon 320 has the potential for restricting guidewire movement within the guidewire lumen 334 or adversely affecting inflation/deflation characteristics of the balloon 320. Thus, the anti-rotation feature of the two hubs 308 and 316 can be advantageous in certain embodiments. The anti-rotation features could include mechanisms such as, but not limited to, one or more keyed tab on the dilator hub 316 and one or more corresponding keyed slot in the sheath hub 308.

In the illustrated embodiment, axial separation motion between the dilator hub 316 and the sheath hub 308 easily disengages the two hubs 308 and 316 while rotational relative motion is prevented by the sidewalls of the tabs and slots. A draft angle on the sidewalls of the tabs and the slots further promotes engagement and disengagement of the anti-rotation feature. In another embodiment, the sheath hub 308 is releaseably affixed to the dilator hub 316 so the two hubs 308 and 316 are coaxially aligned and prevented from becoming inadvertantly disengaged or separated laterally. In this embodiment, the two hubs 308 and 316 are connected at a minimum of 3 points, which prevent lateral relative motion in both of two substantially orthogonal axes. In a preferred embodiment, the two hubs 308 and 316 are engaged substantially around their full 360-degree perimeter. Manual pressure is sufficient to snap or connect the two hubs 308 and 316 together as well as to separate the two hubs 308 and 316.

In another embodiment, the distal end of the sheath hub 308 is configured to taper into the sheath tubing 306 at the distal taper 344 so that the sheath hub 308 distal end 344 and the proximal end of the sheath tubing 306 can be advanced, at least partly, into the urethra or urethral meatus without causing tissue damage. The sheath hub 308 serves as the handle for the sheath 300 and is generally a cylinder of revolution with certain changes in outside diameter moving from distal to proximal end. In the illustrated embodiment, the distal facing surface 340 of the sheath hub 308 can define a cone tapering inward moving increasingly distally. The cone, in longitudinal cross-section, can be characterized by two exterior walls, symmetrically disposed about a centerline, each of said exterior walls being curvilinear and describing a concave outline. In a preferred embodiment, the exterior outline of the distal surface 340 of the sheath hub 308 can describe a linear outline, with surfaces running generally parallel to the longitudinal axis of the sheath tubing 306 and other surfaces running generally perpendicular to the longitudinal axis of the sheath tubing 306. In this preferred embodiment, there are no curvilinear axial cross-sectional outlines except at regions of fillets or other rounding to substantially eliminate any sharp edges that could cut through gloves or fingers. The proximally facing surface 342 of the sheath hub 308 can be curvilinear and flared with a longitudinal cross-section outline appearing like the internal surface of a bell, such shape acting as a funnel for instrumentation. In this embodiment, the axial cross-sectional view of the distally facing surface 342 describes two interior walls, symmetrically disposed about a centerline, each of the walls being convex when viewed from the proximal end of the sheath 300. In a preferred embodiment, the proximally facing surface 342 of the sheath hub 308 can appear substantially linear with edges that are oriented substantially perpendicular to the longitudinal axis of the sheath tubing 306. The access through the proximal surface 342 of the sheath hub 308 to the inner lumen of the sheath 300, can be curvilinear and flared, or it can be linear and describe a lumen that is generally parallel to the longitudinal axis. In another embodiment, the access port through the proximal end 342 of the sheath hub 308 can comprise a straight taper, such as a 6 percent Luer taper to allow for sealing with other devices inserted therein or to allow for ease of device insertion. The amount of end taper can vary between 1½ degrees and 20 degrees between each side and the longitudinal axis of the sheath 300. The maximum outer diameter of the sheath hub 308 can be between 0.25 and 2.0 inches, with a preferred range of between 0.5 and 1.0 inches. The sheath hub 308 can be sized so that at least half a finger diameter is cradled by each side of the flange of the hub 308. The distally facing surface 340 of the sheath hub 308 can furthermore be shaped to have substantially the same curve radius as a finger, so as to be received, or grasped, between two fingers of the hand, cigarette style, like the technique used for control of cystoscopes. In another embodiment, the sheath hub 308 can be sized and configured to be grasped between a thumb and finger, like a pencil or catheter, where there are no features or curves on the distally facing surface 340 of the sheath hub 308 to approximately match or conform to the shape or diameter of two fingers.

In the illustrated embodiment of FIG. 3A, the distal end 304 of the device comprises the catheter shaft 318 and the dilatation balloon 320. The catheter hub 316 may removably lock onto the sheath hub 308 to provide increased integrity to the system and maintain longitudinal relative position between the catheter shaft 318 and the sheath tubing 322 and 306. The catheter hub 316 can have a taper leading from the proximal outside end into any internal or through lumens. The catheter shaft 318 and the balloon 320 are slidably received within the proximal sheath tube 306. The catheter shaft 318 and balloon 320 are slidably received within the distal sheath tube 322 when the distal sheath tube 322 is radially expanded but are frictionally locked within the distal sheath tube 322 when the tube 322 is radially collapsed. The outside diameter of the distal sheath tube 322 ranges from about 4 French to about 16 French in the radially collapsed configuration with a preferred size range of about 5 French to about 10 French. The outside diameter is an important parameter for introduction of the device. Once expanded, the distal sheath tube 322 has an inside diameter ranging from about 8 French to about 20 French. In many applications, the inside diameter is more important than the outside diameter once the device has been expanded. The wall thickness of the sheath tubes 306 and 322 can range from about 0.002 to about 0.030 inches with a preferred thickness range of about 0.005 to about 0.020 inches.

FIG. 3B illustrates a cross-sectional view of the sheath 300 of FIG. 3A wherein the balloon 320 has been inflated causing the sheath tube 322 at the distal end 304 to expand and unfold the longitudinal creases or folds 328. Preferably, the distal sheath tube 322 has the properties of being able to bend or yield, especially at crease lines, and maintain its configuration once the forces causing the bending or yielding are removed. The proximal sheath tube 306 is can be affixed to the sheath hub 308 by insert molding, bonding with adhesives, welding, or the like. As mentioned above, the balloon 320 can be been inflated by pressurizing the annulus between the inner tubing 318 and the outer tubing 324 by application of an inflation device at the inflation port 330 which is integral to, bonded to, or welded to the catheter hub 316. The pressurization annulus empties into the balloon 320 at the distal end of the outer tubing 324. Exemplary materials for use in fabrication of the distal sheath tube 322 include, but are not limited to, polytetrafluoroethylene (PTFE), fluorinated ethylene polymer (FEP), polyethylene, polypropylene, polyethylene terephthalate (PET), and the like. A wall thickness of 0.008 to 0.012 inches is generally suitable for a device with a 16 French OD while a wall thickness of 0.019 inches is appropriate for a device in the range of 36 French OD. In one embodiment, the resulting through lumen of the sheath 300 is generally constant in French size going from the proximal end 302 to the distal end 304. The balloon 320 can be fabricated by techniques such as stretch blow molding from materials such as polyester, polyamide, irradiated polyethylene, and the like.

FIG. 3C illustrates a side cross-sectional view of the sheath 300 of FIG. 3B wherein the catheter shaft 318, the balloon 320, and the catheter hub 316 have been withdrawn and removed leaving the proximal end 302 and the distal end 304 with a large central lumen capable of holding instrumentation. The sleeve 310 and the sleeve grip 312 have also been removed from the sheath 300. The shape of the distal sheath tube 322 may not be entirely circular in cross-section, following expansion, but it is capable of carrying instrumentation the same size as the round proximal tube 306. Because it is somewhat flexible and further is able to deform, the sheath 300 can hold noncircular objects where one dimension is even larger than the round inner diameter of the sheath 300. The balloon 320 is preferably deflated prior to removing the catheter shaft 318, balloon 320 and the catheter hub 316 from the sheath 300.

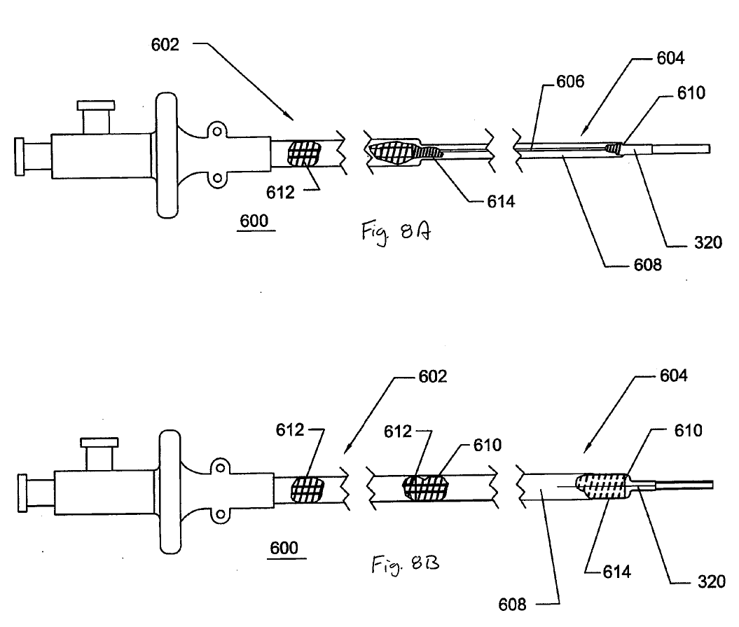

Referring to FIG. 8A, in one embodiment, a proximal reinforcing layer 612 embedded within the proximal sheath tube 602, which is a composite structure, preferably formed from an inner and outer layer. The proximal reinforcing layer 612 can be a coil, braid, or other structure that provides hoop strength to the proximal sheath tube 602. The proximal reinforcing layer 612 can be fabricated from metals such as, but not limited to, stainless steel, titanium, nitinol, cobalt nickel alloys, gold, tantalum, platinum, platinum iridium, and the like. The proximal reinforcing layer 612 can also be fabricated from polymers such as, but not limited to, polyamide, polyester, and the like. Exemplary polymers include polyethylene naphthalate, polyethylene terephthalate, Kevlar, and the like. The proximal reinforcing layer 612, if it comprises metal, preferably uses metal that has been spring hardened and has a spring temper.

Further referring to FIG. 8A, the distal sheath tube 604 is constructed from a composite construction similar to that of the proximal sheath tube 602. The distal reinforcing structure 610, however, is preferably not elastomeric but is malleable. The distal reinforcing structure 610 is preferably a coil of flat or round wire embedded between the inner layer 614 and the outer layer 608. The crease or fold 606 runs longitudinally the length of the distal sheath tube 604 and is the structure that permits the distal sheath tube 604 to be compacted to a smaller diameter than its fully expanded configuration. There may be one fold 606, or a plurality of folds 606. The number of folds 606 can range between 1 and 20, and preferably between 1 and 8, with the sheath tubing 604 bendability and diameter having an influence on the optimal number of folds 606.

The construction of the distal sheath tube 604 can comprise a coil of wire with a wire diameter of 0.001 to 0.040 inches in diameter and preferably between 0.002 and 0.010 inches in diameter. The coil can also use a flat wire that is 0.001 to 0.010 inches in one dimension and 0.004 to 0.040 inches in the other dimension. Preferably, the flat wire is 0.001 to 0.005 inches in the small dimension, generally oriented in the radial direction of the coil, and 0.005 to 0.020 inches in width, oriented perpendicular to the radial direction of the coil. The outer layer 608 has a wall thickness of 0.001 to 0.020 inches and the inner layer 614 has a wall thickness of between 0.001 and 0.010 inches. The wire used to fabricate the coil can be fabricated from annealed materials such as, but not limited to, gold, stainless steel, titanium, tantalum, nickel-titanium alloy, cobalt nickel alloy, and the like. The wire is preferably fully annealed. The wires can also comprise polymers or non-metallic materials such as, but not limited to, PET, PEN, polyamide, polycarbonate, glass-filled polycarbonate, carbon fibers, or the like. The wires of the coil reinforcement can be advantageously coated with materials that have increased radiopacity to allow for improved visibility under fluoroscopy or X-ray visualization. The radiopaque coatings for the coil reinforcement may comprise gold, platinum, tantalum, platinum iridium, and the like. The mechanical properties of the coil are such that it is able to control the configuration of the fused inner layer 614 and the outer layer 608. When the reinforcing layer 610 is folded to form a small diameter, the polymeric layers, which can have some memory, do not generate significant or substantial springback. The sheath wall is preferably thin so that it any forces it imparts to the tubular structure are exceeded by those forces exerted by the malleable distal reinforcing layer. Thus, a peel away or protective sleeve is useful but not necessary to maintain the collapsed sheath configuration.

The inner layer 614 and the outer layer 608 preferably comprise some elasticity or malleability to maximize flexibility by stretching between the coil segments. Note that the pitch of the winding in the distal reinforcing layer 614 does not have to be the same as that for the winding in the proximal reinforcing layer 612 because they have different functionality in the sheath 600.

FIG. 8B illustrates a cutaway sectional view of the sheath 600 of FIG. 6A following expansion by the balloon 320. The proximal sheath tube 602 has not changed its diameter or configuration and the reinforcing layer 612 is likewise unchanged in configuration. The distal tube 604 has become expanded diametrically and the crease or fold 606 of FIG. 8A is now substantially removed. In the illustrated embodiment, due to stress hardening of the reinforcing layer and residual stress in the folded inner layer 614 and outer layer 608, some remnant of the fold 606 may still exist in the distal tube 604. The expansion of the sheath 600 in this configuration can be accomplished using a balloon 320 with an internal pressure ranging between 3 atmospheres and 25 atmospheres. Not only does the balloon 320 impart forces to expand the distal sheath tube 604 against the strength of the reinforcing layer 610 but it also should preferably overcome any inward radially directed forces created by the surrounding tissue. In an exemplary configuration, a sheath 600 using a flat wire coil reinforcing layer 610 fabricated from fully annealed stainless steel 304V and having dimensions of 0.0025 inches by 0.010 inches and having a coil pitch of 0.024 inches is able to fully expand, at a 37-degree Centigrade body temperature, to a diameter of 16 French with between 4 and 7 atmospheres pressurization. The inner layer 614 is polyethylene with a wall thickness of 0.003 to 0.005 inches and the outer layer 608 is polyethylene with a wall thickness of 0.005 to 0.008 inches. The sheath is now able to form a path of substantially uniform internal size all the way from the proximal end to the distal end and to the exterior environment of the sheath at both ends. Through this path, instrumentation may be passed, material withdrawn from a patient, or both. A sheath of this construction is capable of bending through an inside radius of 1.5 cm or smaller without kinking or becoming substantially oval in cross-section.

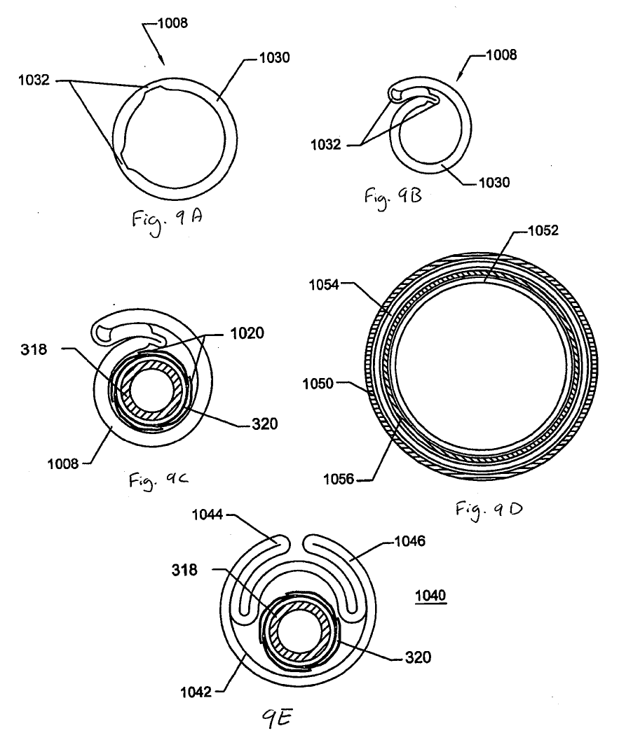

FIG. 9A illustrates a lateral cross-section of an embodiment of the distal tubing 1008, which can be used in combination with the sheath embodiments described above. The distal tubing, in this embodiment, is extruded or formed with thin areas 1032 and normal wall 1030. The illustrated embodiment shows two thin areas 1032 prior to folding. The spacing and magnitude of the thick and thin areas do not necessarily have to be uniformly placed or equally sized. The thin areas can be used to enhance the ability to form tight folds for diameter reduction.

FIG. 9B illustrates the distal tubing 1008 of FIG. 9B after it has been folded longitudinally. Other folds, including Napster™-type styles, star shapes, clover-leafs, folded “W”s, and the like, are also possible. Such profiling can be performed on tubing fabricated from materials such as, but not limited to, polyethylene, PTFE, polyurethane, polyimide, polyamide, polypropylene, FEP, Pebax, Hytrel, and the like, at the time of extrusion. The distal tubing 1008 would then be used, as-is, or it would be built up onto a mandrel with other layers as part of a composite tube. The composite tube can include coil, braid, or stent reinforcement. The thin areas 1032 facilitate tight folding of the layer 1008 and minimize the buildup of stresses and strains in the material that might prevent it from fully recovering to a round shape following unfolding.

FIG. 9C illustrates a lateral cross section of another embodiment of the distal end of the sheath 600. In the illustrated embodiment, the balloon 320 has been folded to form four longitudinal creases, furls, or pleats 1020. The dilator shaft 318 remains in place in the center of the balloon 320 and is fluidically sealed to the balloon 320 at the distal end of said balloon 320. The compressed sheath covering 1008 surrounds the folded balloon 320. When the balloon 320 is expanded under pressure from an external pressure source, the balloon expands the sheath covering 1008 to a larger diameter. The sheath covering 1008 maintains that configuration held in place by the malleable sheath reinforcement or by the malleable nature of the unitary sheath covering 1008, should a separate reinforcement not be used.

FIG. 9D illustrates a lateral cross-section of an embodiment of a sheath tube comprising an inner layer 1052, a reinforcing layer 1056, an elastomeric layer 1054, and an outer layer 1050. The elastomeric layer 1054 can be disposed outside the reinforcing layer 1056, inside the reinforcing layer 1056, or both inside and outside the reinforcing layer 1056. The elastomeric layer 1054 is fabricated from silicone elastomer, thermoplastic elastomer such as C-Flex™, a trademark of Concept Polymers, polyurethane, or the like. The hardness of the elastomeric layer 1054 can range from Shore 10A to Shore 90A with a preferred range of Shore 50A to Shore 70A. The inner layer 1052 and the outer layer 1050 are fabricated from lubricious materials such as, but not limited to, polyethylene, polypropylene, polytetrafluoroethylene, FEP, materials as described in FIG. 8A, or the like. The inner layer 1052 and the outer layer 1050 can have a thickness ranging from 0.0005 inches to 0.015 inches with a preferred range of 0.001 to 0.010 inches. The elastomeric layer 1054 can range in thickness from 0.001 inches to 0.015 inches with a preferred range of 0.002 to 0.010 inches. The reinforcing layer 1056 is as described FIG. 6A. This construction is beneficial for both the proximal non-expandable region and the distal expandable region of the sheath. In an embodiment, the C-Flex thermoplastic elastomer is used for the elastomeric layer 1054 because it fuses well to the polyethylene exterior layer 1050. This embodiment provides for improved kink resistance, improved bendability, and reduced roughness or bumpiness on the surface of the sheath where the elastomeric layer 1054 shields the reinforcing layer 1056. This embodiment provides for a very smooth surface, which is beneficial on both the interior and exterior surfaces of the sheath.

FIG. 9E illustrates a lateral cross-sectional view of an embodiment of an expandable sheath distal section 1040. The sheath distal section 1040 comprises a dilator tube 318, a dilator balloon, 320, and an outer sheath covering 1042, further comprising a first fold 1044 and a second fold 1046. For sheaths with a wall 1042 thickness of about 0.008 to 0.020, it is useful to fold the sheath covering 1042 into two folds 1044 and 1046 if the inside diameter of the expanded sheath ranges greater than 12 French. If the inside diameter of the expanded sheath covering 1042 is less than about 12 French, and sometimes when the sheath covering 1042 is substantially equal to 12 French, it is preferred to have only a single fold, either 1044 or 1046. If the diameter of the sheath covering 1042 is greater than 18 French, or the wall thickness of the sheath covering 1042 is less than the range of about 0.008 to 0.020 inches, or both, additional folds can be added.

One embodiment of the invention comprises a transluminal access system for providing minimally invasive access to anatomically proximal structures. The system includes an access sheath comprising an axially elongate tubular body that defines a lumen extending from the proximal end to the distal end of the sheath. A hub is affixed to the proximal end of the access sheath. The hub is generally non-expandable and prevents trauma if the proximal end of the sheath tubing migrates into the body lumen (for example, the urethra).

In another embodiment of the invention, a transluminal access sheath assembly for providing minimally invasive access comprises an elongate tubular member having a proximal end and a distal end and defining a working inner lumen. In this embodiment, the sheath has a hub affixed to its proximal end. The hub has a proximally facing end, and a distally facing end. The hub is configured with lateral cross-sectional profile that comprises straight lines and angles without any curving. The proximally facing end of the hub can comprise a lip for reversible engagement with a hub affixed to a dilator or obturator. In another embodiment, the hub diameter is smaller than V₂ the diameter of the larger of the index finger or the middle finger. In another embodiment, the distally facing surface comprises a convex curvature, in longitudinal cross-section.

In each of the embodiments, the proximal end of the access assembly, apparatus, or device is preferably fabricated as a structure that is flexible, resistant to kinking, and further retains both column strength and torqueability. Such structures include tubes fabricated with coils or braided reinforcements and preferably comprise inner walls that prevent the reinforcing structures from protruding, poking through, or becoming exposed to the inner lumen of the access apparatus. Such proximal end configurations may be single lumen, or multi-lumen designs, with a main lumen suitable for instrument or obturator passage and additional lumens being suitable for control and operational functions such as balloon inflation. Such proximal tube assemblies can be affixed to the proximal end of the distal expandable segments described heretofore. In an embodiment, the proximal end of the catheter includes an inner layer of thin polymeric material, an outer layer of polymeric material, and a central region comprising a coil, braid, stent, plurality of hoops, or other reinforcement. It is beneficial to create a bond between the outer and inner layers at a plurality of points, most preferably at the interstices or perforations in the reinforcement structure, which is generally fenestrated. Such bonding between the inner and outer layers causes a braided structure to lock in place. In another embodiment, the inner and outer layers are not fused or bonded together in at least some, or all, places. When similar materials are used for the inner and outer layers, the sheath structure can advantageously be fabricated by fusing of the inner and outer layer to create a uniform, non-layered structure surrounding the reinforcement. The polymeric materials used for the outer wall of the jacket are preferably elastomeric to maximize flexibility of the catheter. The polymeric materials used in the composite catheter inner wall may be the same materials as those used for the outer wall, or they may be different. In another embodiment, a composite tubular structure can be co-extruded by extruding a polymeric compound with a braid or coil structure embedded therein. The reinforcing structure is preferably fabricated from annealed metals, such as fully annealed stainless steel, titanium, or the like. In this embodiment, once expanded, the folds or crimps can be held open by the reinforcement structure embedded within the sheath, wherein the reinforcement structure is malleable but retains sufficient force to overcome any forces imparted by the sheath tubing.

In an embodiment of the invention, it cam be beneficial that the sheath comprise a radiopaque marker or markers. The radiopaque markers may be affixed to the non-expandable portion or they may be affixed to the expandable portion. Markers affixed to the radially expandable portion preferably do not restrain the sheath or catheter from radial expansion or collapse. Markers affixed to the non-expandable portion, such as the catheter shaft of a balloon dilator may be simple rings that are not radially expandable. Radiopaque markers include shapes fabricated from malleable material such as gold, platinum, tantalum, platinum iridium, and the like. Radiopacity can also be increased by vapor deposition coating or plating metal parts of the catheter with metals or alloys of gold, platinum, tantalum, platinum-iridium, and the like. Expandable markers may be fabricated as undulated or wavy rings, bendable wire wound circumferentially around the sheath, or other structures such as are found commonly on stents, grafts or catheters used for endovascular access in the body. Expandable structures may also include dots or other incomplete surround shapes affixed to the surface of a sleeve or other expandable shape. Non-expandable structures include circular rings or other structures that completely surround the catheter circumferentially and are strong enough to resist expansion. In another embodiment, the polymeric materials of the catheter or sheath, including those of the sheath composite wall, may be loaded with radiopaque filler materials such as, but not limited to, bismuth salts, or barium salts, or the like, at percentages ranging from 1% to 50% by weight in order to increase radiopacity.

In order to enable radial or circumferential expansive translation of the reinforcement, it may be beneficial not to completely bond the inner and outer layers together, thus allowing for some motion of the reinforcement in translation as well as the normal circumferential expansion. Regions of non-bonding may be created by selective bonding between the two layers or by creating non-bonding regions using a slip layer fabricated from polymers, ceramics or metals. Radial expansion capabilities are important because the proximal end needs to transition to the distal expansive end and, to minimize manufacturing costs, the same catheter may be employed at both the proximal and distal end, with the expansive distal end undergoing secondary operations to permit radial or diametric expansion.

In another embodiment, the distal end of the catheter is fabricated using an inner tubular layer, which is thin and lubricious. This inner layer is fabricated from materials such as, but not limited to, FEP, PTFE, polyamide, polyethylene, polypropylene, Pebax, Hytrel, and the like. Radiopaque filler materials can be added to the polymer inner layer during extrusion to enhance visibility under fluoroscopy. The reinforcement layer comprises a coil, braid, stent, or plurality of expandable, foldable, or collapsible rings, which are generally malleable and maintain their shape once deformed. Preferred materials for fabricating the reinforcement layer include but are not limited to, stainless steel, tantalum, gold, platinum, platinum-iridium, titanium, nitinol, and the like. The materials are preferably fully annealed or, in the case of nitinol, fully martensitic. The outer layer is fabricated from materials such as, but not limited to, FEP, PTFE, polyamide, polyethylene, polypropylene, polyurethane, Pebax, Hytrel, and the like. The inner layer is fused or bonded to the outer layer through holes in the reinforcement layer to create a composite unitary structure. The structure is crimped radially inward to a reduced cross-sectional area. A balloon dilator is inserted into the structure before crimping or after an initial crimping and before a final sheath crimping. The balloon dilator is capable of forced expansion of the reinforcement layer, which provides sufficient strength necessary to overcome any forces imparted by the polymeric tubing.

Another embodiment of the invention comprises a method of providing transluminal access. The method comprises inserting a cystoscope into a patient, transurethrally, into the bladder. Under direct optical visualization, fluoroscopy, MRI, or the like, a guidewire is passed through the instrument channel of the cystoscope and into the bladder. The guidewire is manipulated, under the visual control described above, into the ureter through its exit into the bladder. The guidewire is next advanced to the appropriate location within the ureter. The cystoscope is next removed, leaving the guidewire in place. The ureteral access sheath is next advanced over the guidewire transurethrally so that its distal tip is now resident in the ureter or the kidney. The ureteral access sheath is handled by the operator using the index finger and thumb of one hand. The position of the guidewire is maintained carefully so that it does not come out of the ureter and fall into the bladder. The removable dilator comprises the guidewire lumen, and is used to guide placement of the access sheath into the urinary lumens. Expansion of the distal end of the access sheath from a first smaller diameter cross-section to a second larger diameter cross-section is next performed, using the balloon dilator. The balloon dilator is subsequently removed from the sheath to permit passage of instruments that would not normally have been able to be inserted into the ureter due to the presence of strictures, stones, or other stenoses. The method further optionally involves releasing the elongate tubular body from a constraining tubular jacket, removing the expandable member from the elongate tubular body; inserting appropriate instrumentation, and performing the therapeutic or diagnostic procedure. Finally, the procedure involves removing the elongate tubular body from the patient. Once the sheath is in place, the guidewire may be removed or, preferably, it may be left in place. Alternatively, a second guidewire, or safety wire, can be introduced into the ureter and be placed alongside or through the sheath.

In one embodiment, where the transluminal access sheath is used to provide access to the upper urinary tract, the access sheath may be used to provide access by tools adapted to perform biopsy, urinary diversion, stone extraction, antegrade endopyelotomy, and resection of transitional cell carcinoma and other diagnostic or therapeutic procedures of the upper urinary tract or bladder. Other applications of the transluminal access sheath include a variety of diagnostic or therapeutic clinical situations, which require access to the inside of the body, through either an artificially created, percutaneous access, or through another natural body lumen.

The present invention may be embodied in other specific forms without departing from its spirit or essential characteristics. For example, the sheath may include instruments affixed integrally to the interior central lumen of the mesh, rather than being separately inserted, for performing therapeutic or diagnostic functions. The hub may comprise tie downs or configuration changes to permit attaching the hub to the skin of the patient. The embodiments described herein further are suitable for fabricating very small diameter catheters, microcatheters, or sheaths suitable for cardiovascular or neurovascular access. These devices may have collapsed diameters less than 3 French (1 mm) and expanded diameters of 4 to 8 French. Larger devices with collapsed diameters of 16 French and expanded diameters of 60 French or larger are also possible. Such large devices may have orthopedic or spinal access applications, for example. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or subcombinations of the specific features and aspects of the embodiments may be made and still fall within the scope of the invention. Accordingly, it should be understood that various features and aspects of the disclosed embodiments can be combine with or substituted for one another in order to form varying modes of the disclosed invention. Thus, it is intended that the scope of the present invention herein disclosed should not be limited by the particular disclosed embodiments described above, but should be determined only by a fair reading of the claims that follow

What is claimed is:

1. A transluminal access sheath for insertion into a urethra by a person having a pair of adjacent fingers, the access sheath comprising:
an elongate tube having a lumen extending between a proximal end and a distal end, the elongate tube having a distal portion and a proximal portion;
a removable inner member disposed within the lumen of the elongate tube;
a hub coupled to the proximal end of the elongate tube, the hub comprising a distally facing surface and a proximally facing surface, the distally facing surface forming at least in part a straight cone, sized and configured to receive adjacent fingers of the user, the proximally facing surface forming a straight taper configured to funnel instrumentation into the lumen.

2. The transluminal sheath of claim 1 wherein the sheath hub is configured at its distal end to slip into the urethra and to allow the proximal end of the sheath tube to slip into the urethra.

3. The transluminal sheath of claim 1 wherein the sheath hub is configured with a substantially linear outline when viewed in axial cross-section.

4. The transluminal sheath of claim 1 wherein the sheath hub has a diameter that is less than ½ the diameter of an average adult finger, the shape of the distally facing side of the hub is not curved to a radius substantially the same as a finger, and wherein the hub is not receivable by adjacent fingers.

5. The transluminal sheath of claim 1, wherein the distal portion of the elongate tube is expandable from a first, smaller diameter to a second, greater diameter.

6. The transluminal sheath of claim 5, wherein the proximal portion of the elongate tube is substantially non-expandable and which is affixed at its distal end to a proximal end of the expandable distal portion.

7. The transluminal sheath of claim 6, wherein the inner member comprise a dilator that can be used to expand the distal portion of the access sheath

8. The transluminal sheath of claim 1 wherein the proximally facing side of the sheath hub is releasably affixed to an inner member hub, which covers the proximally facing side of the sheath hub.

9. The transluminal sheath of claim 1, wherein the inner member hub further comprises a dilator inflation port and a guidewire lumen.
